(19) United States
(12) Patent Application Publication
(10) Pub. No.: US 2006/0084876 A1
(43) Pub. Date: Apr. 20, 2006

(54) Method for reducing auto-fluorescence signals in confocal Raman microscopy

(76) Inventors: Chil Hwan Oh, Seoul (KR); Dae Gab Gweon, Daejeon (KR); Hyo Jin Kim, Seoul (KR); Jeung Hee Park, Sungnam-si (KR); Jae Bum Choo, Ansan-si (KR); Hoi Eil Chung, Seoul (KR); Jung Hyun Choi, Anyang-si (KR)

Correspondence Address: CLARK & ELBING LLP, 101 FEDERAL STREET, BOSTON, MA, 02110, US

(21) Appl. No.: 11/254,927

(22) Filed: Oct. 20, 2005

(30) Foreign Application Priority Data
Oct. 20, 2004 (KR) 2004-0083980

(51) Int. Cl. A61B 6/00

(52) U.S. Cl. 600/476

(57) ABSTRACT

The present invention relates to a method for reducing auto-fluorescence signals from a sample tissue in confocal Raman microscopy and to a method for diagnosing skin cancers using the same method. Raman spectroscopy has strong potential for providing non-invasive diagnosis of skin cancer. Auto-fluorescence signals from tissues, which interfere with the Raman signals, were greatly reduced using a confocal slit adjustment. Distinct Raman band differences between normal and BCC tissues for the amide I mode, the amide III mode and the PO₂⁻ symmetric stretching mode, showed that the present invention has strong potential for use as a dermatological diagnostic tool without the need for statistical treatment of spectral data. It was also possible to precisely differentiate BCC tissue from surrounding non-cancerous tissue using the confocal Raman depth profiling technique. According to the present invention, confocal Raman microscopy can provide a novel method for dermatological diagnosis since direct observations of spectral differences between normal and BCC tissues are possible.

### CROSS-REFERENCE TO RELATED PATENT APPLICATION

This application claims the benefit of Korean Patent Application No. 10-2004-0083980, filed on Oct. 20, 2004, in the Korean Intellectual Property Office, the disclosure of which is incorporated herein in its entirety by reference

### BACKGROUND OF THE INVENTION

1. Field of the Invention

The present invention relates to a method for reducing auto-fluorescence signals from a sample tissue in confocal Raman microscopy and to a method for diagnosing skin cancers using the same method.

2. Description of the Related Art

Advances in spectroscopic technology over the past few decades have had a significant impact on the area of cancer research. Technologies such as FT-IR, Raman and confocal fluorescence microscopy have been used to elucidate the origin and progression of cancer (Jackson, M. Faraday Discuss 2004, 126, 1-18; Yano et al. Analyt Biochem 2000, 287, 218-225). These technologies have also been used for the early detection of cancer and biochemical changes in cells and tissues causing cancer (Shafer-Peltier et al., J Raman Spectrosc 2002, 33, 552-563; Ling et al., Appl Spectrosc 2002, 56, 570-573). Among these spectroscopic techniques, Raman spectroscopy has attracted considerable attention for medical diagnosis since it is non-destructive, does not require a sample preparation process and provides detailed information about the molecular structure of normal and diseased tissues.

Recently, the incidence of skin cancer has dramatically increased due to the excessive exposure of skin to UV radiation caused by ozone layer destruction, environmental contamination and so on. If detected early, skin cancer has a cure rate of 100%. Unfortunately, early detection is difficult because diagnosis is still based on morphological inspection by a pathologist. There are two common skin cancers: basal cell carcinoma (BCC) and squamous cell carcinoma (SCC). Both BCC and SCC are non-melanoma skin cancers and BCC is the most common skin neoplasm. The accurate detection of BCC has attracted much attention from clinical dermatologists since it is difficult to distinguish BCC tissue from surrounding non-cancerous tissue (Caspers et al., J Invest Dermatol 2001, 116, 434-442; Bakker Schut et al., Anal Chem 2000, 72, 6010-6018). The routine diagnostic technique used for the detection of BCC is pathological examination of biopsy samples. This involves removal of tissue from suspected abnormal areas, which is then sliced and stained to enable the pathologist to identify morphological abnormalities. This method relies upon a subjective judgment, which is dependent on the level of experience of the individual pathologist and can lead to the excessive biopsy of tissues. Thus, a fast and accurate diagnostic technique for the initial screening and selection of lesions for further biopsy is needed.

Raman spectroscopy has the potential to resolve this problem. It can be applied to provide a more accurate medical diagnosis to distinguish BCC tissue from surrounding normal tissue. Recently, several research groups carried out BCC detection using Raman spectroscopy. Gniadecka et al. and Nunes et al. used FT-Raman spectroscopy to distinguish between BCC and surrounding normal tissues (Gniadecka et al., J Invest Dermatol 2004, 122, 443-449; Nunes et al., Spectroscopy 2003, 17, 597-602). Nijssen et al. demonstrated pseudo-color Raman maps based on FT-Raman spectra obtained from a frozen tissue section of BCC (Nijssen et al., J Invest Dermatol 2002,119, 64-69). In this work, they showed that the pseudo-color Raman map is closely related to the microscopic image of the hematoxylin and eosin (H&E) stained sample. In previous studies using FT-Raman spectroscopy, a long wavelength excitation laser (1064 nm Nd:YAG laser or 850 nm titanium-sapphire laser) has been applied to minimize auto-fluorescence from skin tissue. However, the longer wavelength laser has poor Raman scattering intensities compared to the shorter excitation wavelength laser. As a result, previously reported Raman spectra on BCC tissues show poor signal-to-noise ratios and require statistical treatment of spectroscopic data, such as principal component analysis (PCA) (Nunes et al., Spectroscopy 2003,17, 597-602), K-mean clustering analysis (KCA) (Caspers et al., Biophys J 2003, 85, 572-580), and neural networks (Gniadecka et al., J Invest Dermatol 2004, 122, 443-449) to differentiate between Raman signals of cancerous and normal tissues. For the practical application of cancer diagnosis, however, direct observation of spectral differences without any statistical treatment of spectral data is essentially required.

### SUMMARY OF THE INVENTION

The present invention provides a method for reducing auto-fluorescence signals from a sample tissue, which interfere with the Raman signals, by confocal slit adjustment in confocal Raman microscopy.

The present invention also provides a method for diagnosing skin cancers using confocal Raman microscopy, characterized in that auto-fluorescence signals from a sample tissue, which interfere with the Raman signals, in the confocal Raman microscopy are reduced by confocal slit adjustment.

Raman spectroscopy has strong potential for providing non-invasive diagnosis of skin cancer. In the present invention, confocal Raman microscopy was applied to the dermatological diagnosis for one of the most common skin cancer, basal cell carcinoma (BCC). Tissues were obtained from 10 BCC patients using a routine biopsy and used for confocal Raman measurements. Auto-fluorescence signals from tissues, which interfere with the Raman signals, were greatly reduced using a confocal slit adjustment. Distinct Raman band differences between normal and BCC tissues for the amide I mode, the amide III mode and the PO₂⁻ symmetric stretching mode, showed that this technique has strong potential for use as a dermatological diagnostic tool without the need for statistical treatment of spectral data. It was also possible to precisely differentiate BCC tissue from surrounding non-cancerous tissue using the confocal Raman depth profiling technique. According to the present invention, confocal Raman microscopy can provide a novel method for dermatological diagnosis since direct observations of spectral differences between normal and BCC tissues are possible.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features and advantages of the present invention will become more apparent by describing in detail exemplary embodiments thereof with reference to the attached drawings in which:

FIG. 1 shows microscopic images of H&E stained sections for skin tissue collected from three different patients (a, b and c). Dark blue regions are malignant tissues (BCC) and lighter eosinophilic regions are normal tissues.

FIG. 2 shows Raman spectra of BCC tissue: (a) non-confocal mode Raman spectrum; (b) confocal mode Raman spectrum.

FIG. 3 shows confocal Raman spectra of skin tissue in the 1000-1700 cm⁻¹ region: (a) Raman spectrum of normal tissue; (b) curve-fitting Raman spectrum of BCC tissue.

FIG. 4 shows confocal Raman spectra of normal and malignant tissue from three different patients: (a) normal tissue; (b) BCC tissue.

FIG. 5 shows confocal Raman profile of skin tissue (FIG. 1(a)) at seven different spots with the intervals of 30-40 μm: (a) enlarged microscopic image of H&E stained skin tissue; (b) corresponding Raman spectra at the seven areas indicated in (a).

FIG. 6 shows confocal Raman profile of skin tissue (FIG. 1(b)) at seven different spots with the intervals of 30-40 μm: (a) enlarged microscopic image of H&E stained skin tissue; (b) corresponding Raman spectra at the seven spots indicated in (a).

FIG. 7 shows confocal Raman profile of skin tissue (FIG. 1(c)) at seven different spots with the intervals of 30-40 μm: (a) enlarged microscopic image of H&E stained skin tissue; (b) corresponding Raman spectra at the seven spots indicated in (a).

### DETAILED DESCRIPTION OF THE INVENTION

In the present invention, the confocal Raman technique, using a shorter wavelength argon ion laser at 514.5 nm, was applied to the dermatological diagnosis of skin cancer such as BCC. To effectively remove auto-fluorescence, which strongly interferes with Raman signals, the confocal technique was used. Confocal Raman microscopy has been known to exhibit a superior rejection of fluorescence. This arises from the fact that the laser is focused on such a small point that the laser flux in the sampling volume is high enough to quench fluorescence in a fraction of time often needed in conventional Raman spectroscopy. Second, the excited electron, along with its slit is, can migrate from one molecule to the other so that a molecule outside the focal volume may emit the fluorescence photon. These photons are blocked by the confocal slit. Consistent spectral differences between normal and cancerous tissues were observed for 10 BCC samples investigated using confocal Raman microscopy. Furthermore, confocal Raman depth profiling was performed to differentiate BCC tissue from surrounding non-cancerous tissue; it is important to determine the removed area in a surgical operation. In the present invention, we have found the potential for confocal Raman microscopy to be used as a direct diagnostic tool for pre-cancerous and non-cancerous lesions.

According to an aspect of the present invention, there is provided a method for reducing auto-fluorescence signals from a sample tissue, which interfere with the Raman signals, by confocal slit adjustment in confocal Raman microscopy.

In the aspect of the present invention, the confocal Raman microscopy may be performed using any confocal Raman microscope such as Renishaw 2000 Raman microscope system, which is commercially available, according to a protocol provided by the manufacturer. In confocal Raman microscopes, the Raman spectometer is usually attached to a light microscope. The depth resolution and optical slicing of a sample are provided by a pinhole that restricts the signals emerging from out-of-focus zones. The confocal pinhole may be replaced by a combination of a slit and CCD area.

In the aspect of the present invention, the confocal slit adjustment may be performed by a two-slit confocal arrangement, in which the first confocal slit is set to a width of 10-20 μm and a virtual second slit is created from two pixel rows on the CCD detector, that was aligned perpendicular to the spectrometer slit.

According to another aspect of the present invention, there is provided a method for diagnosing skin cancers using confocal Raman microscopy, which comprises detecting distinct Raman band differences between normal and skin cancer tissues for the amide I mode, the amide III mode or the PO₂⁻ symmetric stretching mode, wherein auto-fluorescence signals from a sample tissue, which interfere with the Raman signals, in the confocal Raman microscopy are reduced by confocal slit adjustment.

In the aspect of the present invention, the skin cancer may be any skin cancer such as basal cell carcinoma (BCC) or squamous cell carcinoma (SCC), preferably, basal cell carcinoma (BCC).

In the aspect of the present invention, the confocal slit adjustment may be performed by a two-slit confocal arrangement, in which the first confocal slit is set to a width of 10-20 μm and a virtual second slit is created from two pixel rows on the CCD detector, that was aligned perpendicular to the spectrometer slit.

In the aspect of the present invention, distinct Raman band differences between normal and skin cancer tissues for the amide I mode, the amide III mode or the PO₂⁻ symmetric stretching mode can be detected without the need for statistical treatment of spectral data. In the amide I mode and the amide III mode, structural changes of protein amide between normal and cancer tissues can be detected. In the PO₂⁻ symmetric stretching mode, structural changes of phospholipids and nucleic acids between normal and cancer tissues can be detected.

In the aspect of the present invention, the distinct Raman band differences between normal and skin cancer tissues may be detected by observing the intensity changes in the 1000-1700 cm⁻¹ region of Raman shift.

In the aspect of the present invention, the distinct Raman band differences may be detected in the 1580-1610 cm⁻¹ region of Raman shift for the amide I mode, in the 1320-1340 cm⁻¹ region of Raman shift for the amide III mode, or in the 1030-1060 cm⁻¹ region of Raman shift for the PO₂⁻ symmetric stretching mode.

Specifically, in the Raman spectra for the amide I mode, the amide I band was found in 1630-1660 cm⁻¹ region for normal tissue, whereas the band (peak) was shifted to the lower frequency in 1580-1610 cm⁻¹ region and its band-width was greatly broadened for BCC tissue. In the Raman spectra for the amide III mode, the amide III band was found in 1290-1310 cm⁻¹ region for normal tissue, whereas the band was shifted to a higher frequency in 1320-1340 cm⁻¹ region and its intensity decreased for BCC tissue. In the Raman spectra for the PO₂⁻ symmetric stretching mode, the PO₂⁻ symmetric stretching mode band was found in 1070-1100 cm⁻¹ region for normal tissue, whereas the band was shifted to a lower frequency in 1030-1060 cm⁻¹ region and its intensity greatly reduced for BCC tissue.

In the aspect of the present invention, the skin cancer tissue may be precisely differentiated from surrounding non-cancerous tissue using the confocal Raman depth profiling technique. The confocal Raman depth profiling technique may be performed by detecting Raman signals with consecutively scanning laser beams having focal scale of 1-2 μm with intervals of 30-40 μm.

The present invention now will be described in greater detail by means of the following examples. The following examples are for illustrative purpose and are not intended to limit the scope of the invention.

### EXAMPLE 1

### Skin Tissue Preparations

Skin tissue samples were obtained from the Dermatologic Department of Korea University Hospital in Korea. BCC tissues used for confocal Raman measurements were obtained from 10 patients using a routine biopsy. Cross-sections 20 μm thick were cut with a microtome at −20° C., and frozen sections were stored in liquid nitrogen before use. Two thin sections were cut for the experiments. One section was used for confocal Raman profiling experiments and the other section was stained with H&E and sent to an expert pathologist for a routine cancer diagnosis. The H&E section was also used as a Raman reference to locate the boundaries between the different skin-layers in the unstained section. In the present paper, characteristic Raman spectra for three different area of BCC tissues will be introduced.

### EXAMPLE 2

### Confocal Raman Measurements

Confocal Raman measurements were performed using a Renishaw 2000 Raman microscope system. A Spectra Physics argon ion laser operating at λ=514.5 nm was used as the excitation source with a laser power of approximately 20 mW. The Rayleigh line was removed from the collected Raman scattering by a holographic notch filter located in the collection path. Raman scattering was detected using a charge-coupled device (CCD) camera at a spectral resolution of 4 cm⁻¹. An additional CCD camera was fitted to an optical microscope to obtain optical images of skin tissue samples. A two-slit confocal arrangement was used to reduce background Raman scattering from the unfocused laser beams. All Raman spectra were measured in the confocal mode to remove out-of-focus signals (Lee et al., J Raman Spectrosc 2003, 34, 737-742). In the Raman system, the function of the pinhole was replaced by the cooperation of the entrance slit and the pixels in the CCD detector. The first confocal slit was set to a width of 15 μm. The signal was then collected from only two pixel rows on the CCD detector, creating a virtual second slit that was aligned perpendicular to the spectrometer slit. In this way, stray background light due to any out-of-focus regions of the skin tissue sample was effectively removed.

### RESULTS AND DISCUSSION

FIG. 1 shows microscopic images of the H&E stained tissues obtained from three different skin cancer patients. Dark regions are BCC tissues and lighter regions are normal tissues. This pathological examination of biopsy samples provides a standard for the Raman spectroscopic identification of BCC. To confirm the necessity of the confocal technique in Raman measurements, the Raman spectra of BCC were measured using two different modes: a non-confocal mode and a confocal mode.

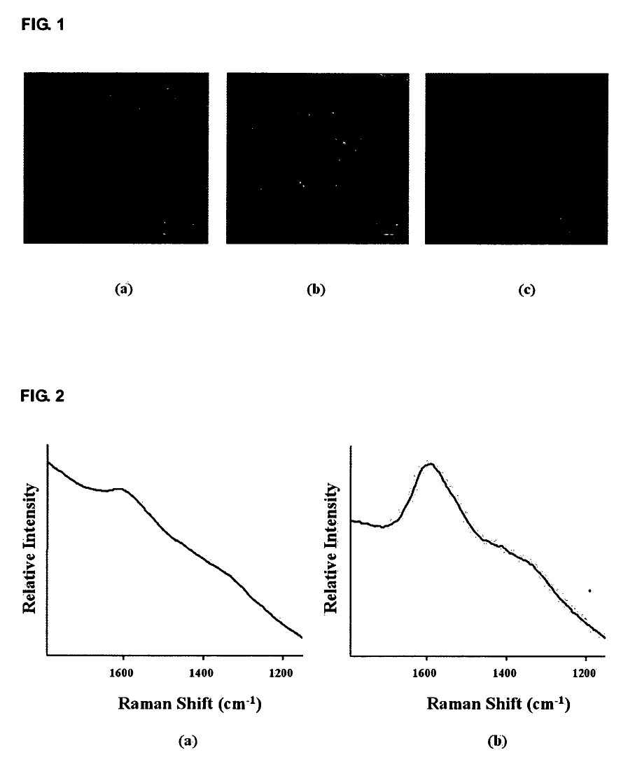

FIG. 2 shows a comparison of the Raman spectra of BCC tissues measured using these two different modes: the non-confocal mode in FIG. 2a and the confocal mode in FIG. 2b. Fluorescence interference was very high in the non-confocal mode Raman spectrum, whereas interference signals from BCC tissues were greatly reduced in the confocal mode spectrum when a 15 μm wide confocal pinhole was used to remove any unfocused scattering signals. Therefore, the confocal technique is very effective at reducing auto-fluorescence signals from tissues.

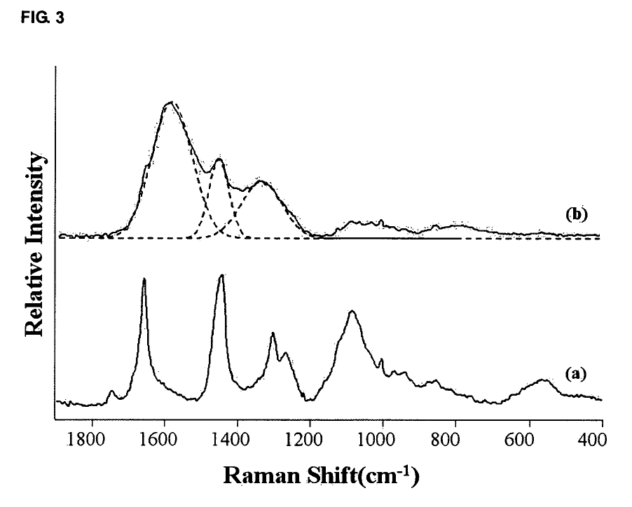

FIG. 3 shows the confocal Raman spectra for the skin sample shown in FIG. 1a. A clear distinction was found between normal and BCC tissues by confocal Raman microscopy. Many differences in these spectra arise from the intensity changes in the 1000-1700 cm⁻¹ region. In previous studies (Nijssen et al., J Invest Dermatol 2002, 119, 64-69; Gniadecka et al., J Invest Dermatol 2004, 122, 443-449; Gniadecka et al., J. Photochem Photobio 1997, 66, 418-423; Gniadecka et al., J. Raman Spectrosc 1997, 28, 125-129; Nunes et al., Spectroscopy 2003,17, 597-602), trivial spectral differences between normal and BCC tissues were found only in the amide III mode at the 1250-1350 cm⁻¹ region and in the PO₂⁻ stretching mode at the 1000-1100 cm⁻¹ region. In our confocal Raman spectra, however, clear spectral differences were found in four characteristic Raman bands in this region. As shown in FIG. 3, it is possible to make a visual diagnosis from the spectra without any statistical treatment. For a more detailed comparison between the two Raman spectra, the Raman bands of BCC tissue were decomposed using Gaussian curve-fitting analysis. The broad overlap of bands in the 1000-1100 cm⁻¹ region was decomposed into three characteristic bands, which are listed in Table 1.

TABLE 1
Observed Confocal Raman Frequencies of
surrounding normal and BCC Tissues

| | Normal tissue | BCC tissue |
|---|---|---|
| Vibrational descriptions | (cm⁻¹) | (cm⁻¹)^{a} |
| Amide I mode | 1656 | 1589 |
| Lipid and protein mode | 1441 | 1450 |
| (CH₂ deformation) | | |
| Amide III mode | 1302 | 1328 |
| Phospholipid and nucleic | 1085 | 1048 |
| acid mode (PO₂⁻sym. stretch) | | |

^{a}Assigned frequencies based on curve-fitting analysis.

In the Raman spectra, the amide I band at 1656 cm⁻¹ was shifted to the lower frequency and its band-width was greatly broadened for BCC tissue, whereas the amide III band at 1302 cm⁻¹ was shifted to a higher frequency and its intensity decreased. These results are similar to previous Raman data reported by Gniadecka et al. (J Invest Dermatol 2004, 122, 443-449; J. Photochem Photobio 1997, 66, 418-423; J. Raman Spectrosc 1997, 28, 125-129), but our confocal spectra show more distinct differences between normal and BCC tissues. The Raman band changes in amide I and amide III modes are closely related to the structural changes in secondary protein between normal and BCC tissues. The CH₂ deformation mode at 1441 cm⁻¹ for lipids and proteins shifted to a higher frequency and its intensity decreased. The PO₂⁻ symmetric stretching mode at 1085 cm⁻¹ for phospholipids and nucleic acids shifted to a lower frequency and its intensity greatly reduced.

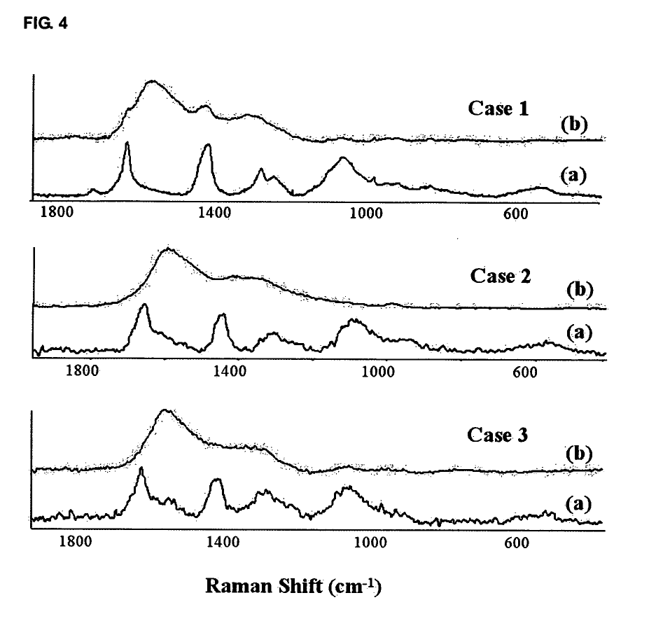

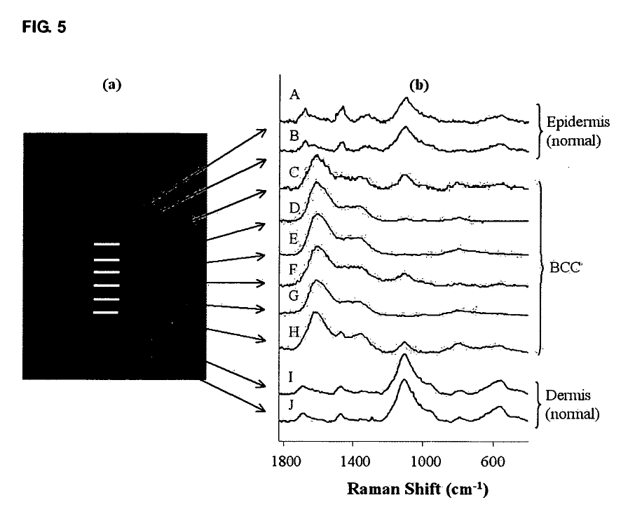

FIG. 4 shows the Raman spectra for normal and BCC skin tissues from the three different patients in FIG. 1. The Raman spectra for normal tissues of three different patients are very similar to each other and also to those previously reported. The Raman spectra of BCC tissues from the three patients also show very good reproducibility. Direct differences between normal and BCC tissues can also be seen (FIG. 4). In particular, the enhancement of the amide III band and the disappearance of the PO₂⁻ stretching band in BCC tissue can be used for the diagnosis of a skin cancer.

Another important factor in dermatological diagnosis is how precisely BCC can be distinguished from surrounding non-cancerous tissue using Raman spectroscopy (Kaminaka et al., J Raman Spectrosc 2001, 32, 139-141; Kaminaka et al., J Raman Spectrosc 2002, 33, 498-502). Raman spectroscopy has the potential to be used as a fast and accurate diagnostic tool for the screening and selection of a biopsy area. In order to confirm this, we performed confocal Raman depth profiling for BCC and surrounding tissue. FIG. 5 shows the enlarged microscopic image of H&E stained tissue in FIG. 1a and its confocal Raman spectra at 10 consecutive spots with intervals of 30-40 μm. The H&E stained image was used as a reference since we could not see the difference between normal and BCC areas with the image of frozen tissues for the Raman measurements. The strong PO₂⁻ symmetric stretching band centered at 1085 cm⁻¹ was observed at the normal epidermis (A and B) and dermis (I and J) areas (FIG. 5). On the other hand, the strong amide I band centered at 1589 cm⁻¹ was observed in the BCC (D, E, F and G) area. On the border between normal and BCC tissues (C and H), intermediate intensities of the PO₂⁻ band were observed.

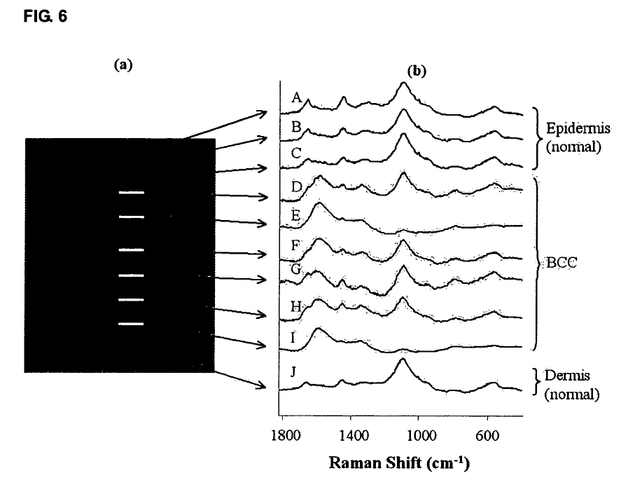

In order to confirm the reproducibility of the confocal Raman technique for dermatological diagnosis, we performed confocal Raman profiling experiments for the other skin tissues in FIG. 1. FIG. 6 shows the Raman profiling data for the second skin tissue sample in FIG. 1b. In this case, very similar patterns of Raman bands to the first case were observed. In the epidermal area (A, B and C), the strong BCC amide I band was not observed and the characteristic strong PO₂⁻ band of the normal tissue was found. For the BCC area (D, E, F, G, H and I), however, strong amide I bands were observed but strong PO₂⁻ bands for normal tissue were also found in some cases (D, F, G and H). When the H&E stained image was compared with the corresponding Raman spectra, it appeared that the BCC had not homogeneously metastasized. The last spectrum (J) in the dermal area shows the characteristic Raman bands of non-cancerous tissue.

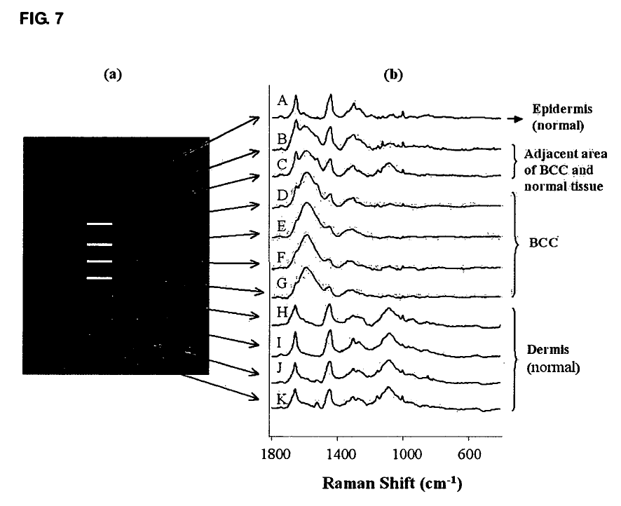

FIG. 7 displays spectra for the sample in FIG. 1c. At the dermal area (A, H, I, J and K), typical Raman spectra of normal tissue were seen, and at the BCC dermis area (D, E, F and G) characteristic Raman bands for BCC tissues were observed. In this sample, however, it is interesting to note that mixed Raman bands were observed on the border (B and C) between normal and BCC tissues. The spectral changes observed form A to D show that the amide I band for the normal tissue has shifted to the band for the BCC tissue. Clear intensity changes in the PO₂⁻ and amide III bands from the normal side to the cancerous side of the tissue can also be seen. These results show that by applying the confocal Raman depth profiling technique to human skin tissue, it is possible to precisely determine cancerous tissue from surrounding non-cancerous tissue.

Raman spectroscopy has been widely used to differentiate BCC tissue from normal surrounding tissue. In earlier studies, the FT-Raman spectroscopic technique, using a long wavelength near-infrared laser, has been applied to minimize auto-fluorescence from human tissues. In these cases, however, the Raman intensity is relatively weak due to a poor scattering efficiency, even though the spectrum has accumulated for a long time. As a result, statistical treatment of collected Raman data, using PCA analysis or neural network, is required to differentiate BCC tissue from normal tissue. In the present work, we found that confocal Raman microscopy, using the 514.5 nm argon ion laser, can be successfully applied to diagnosis of BCC. The out-of-plane auto-fluorescence signals from tissues could be efficiently removed by the confocal technique. In particular, distinct Raman band differences between normal and BCC tissues for the amide I mode and the PO₂⁻ symmetric stretching mode show that this confocal Raman technique has strong potential as a dermatological diagnostic tool without the need for any statistical treatment of data.

A fast and accurate BCC diagnostic tool for the screening and selection of biopsy is important in Moh's micrographic surgery. Therefore, confocal Raman depth profiling could be used to accurately determine the BCC area from healthy surrounding tissue, making it possible to precisely differentiate a cancerous area from surrounding non-cancerous tissues. Mixed amide I bands of normal and BCC tissues around the borderline, as well as irregular patterns of PO₂⁻ bands of normal tissue caused by inhomogeneous metastasis inside the BCC area, can also be detected. We predict that this confocal Raman profiling technique has strong feasibility as a dermatological diagnostic tool.

According to the present invention, auto-fluorescence signals from tissues, which interfere with the Raman signals, can be greatly reduced using a confocal slit adjustment. The present invention has strong potential for use as a dermatological diagnostic tool without the need for statistical treatment of spectral data. It was also possible to precisely differentiate BCC tissue from surrounding non-cancerous tissue using the confocal Raman depth profiling technique.

While the present invention has been particularly shown and described with reference to exemplary embodiments thereof, it will be understood by those skilled in the art that various changes in form and details may be made therein without departing from the spirit and scope of the present invention as defined by the appended claims.

What is claimed is:

1. A method for reducing auto-fluorescence signals from a sample tissue, which interfere with the Raman signals, by confocal slit adjustment in confocal Raman microscopy.

2. The method according to claim 1, wherein the confocal slit adjustment is performed by a two-slit confocal arrangement, in which the first confocal slit is set to a width of 10-20 μm and a virtual second slit is created from two pixel rows on the CCD detector, that was aligned perpendicular to the spectrometer slit.

3. A method for diagnosing skin cancers using confocal Raman microscopy, which comprises detecting distinct Raman band differences between normal and skin cancer tissues for the amide I mode, the amide III mode or the PO₂⁻ symmetric stretching mode, wherein auto-fluorescence signals from a sample tissue, which interfere with the Raman signals, in the confocal Raman microscopy are reduced by confocal slit adjustment.

4. The method according to claim 3, wherein the skin cancer is a basal cell carcinoma (BCC).

5. The method according to claim 3, wherein the distinct Raman band differences between normal and skin cancer tissues are detected by observing the intensity changes in the 1000-1700 cm⁻¹ region of Raman shift.

6. The method according to claim 3, wherein the distinct Raman band differences are detected in the 1580-1610 cm⁻¹ region of Raman shift for the amide I mode, in the 1320-1340 cm⁻¹ region of Raman shift for the amide III mode, or in the 1030-1060 cm⁻¹ region of Raman shift for the PO₂⁻ symmetric stretching mode.

7. The method according to claim 3, wherein the skin cancer tissue is precisely differentiated from surrounding non-cancerous tissue using the confocal Raman depth profiling technique.

8. The method according to claim 7, wherein the confocal Raman depth profiling technique is performed by detecting Raman signals with consecutively scanning laser beams having focal scale of 1-2 μm with intervals of 30-40 μm.
